(19) United States
(12) Patent Application Publication
(10) Pub. No.: US 2011/0264132 A1
(43) Pub. Date: Oct. 27, 2011

(54) MULTI-UTILITARIAN MICROCATHETER SYSTEM AND METHOD OF USE

(75) Inventors: Brian M. Strauss, Trabuco Canyon, CA (US); Jeffrey J. Valko, San Clemente, CA (US); Michael R. Henson, Coto de Caza, CA (US)

(73) Assignee: REVERSE MEDICAL CORPORATION, Irvine, CA (US)

(21) Appl. No.: 12/936,326

(22) PCT Filed: Apr. 3, 2009
(86) PCT No.: PCT/US09/39543

§ 371 Date: Mar. 24, 2011

Related U.S. Application Data
(60) Provisional application No. 61/042,687, filed on Apr. 4, 2008.

(51) Int. Cl. A61M 29/00

(52) U.S. Cl. 606/194

(57) ABSTRACT

A device for performing therapeutic or diagnostic procedures within the cerebrovasculature includes a catheter having a distal portion, a proximal portion and a lumen extending therebetween, the catheter including an expandable region for engaging the vessel wall, thrombus, atheroma, or other structures. The device further includes an elongate stretching member, which can be a guidewire, insertable longitudinally through the lumen of the catheter, the elongate stretching member being configured for stretching at least a portion of the catheter and causing the expandable region to transition from an expanded state to a collapsed state, and wherein the elongate stretching member is retracted proximally relative to the catheter causes the expandable region to transition from the radially collapsed state to a radially, or laterally expanded state.

### RELATED APPLICATION

This patent application claims priority to U.S. Provisional Patent Application No. 61/042,687 filed Apr. 4, 2009, the entire disclosure of which is expressly incorporated herein by reference.

### FIELD OF THE INVENTION

The field of the invention generally relates to devices and methods for protecting cerebral vessels and brain tissue during endovascular treatment. More particularly, the field of the invention pertains to devices and methods for interventional neuroradiology.

### BACKGROUND OF THE INVENTION

Thromboembolic disorders, such as occlusive stroke, pulmonary embolism, myocardial infarct, peripheral thrombosis, atherosclerosis, and the like, affect many people. These disorders are a major cause of morbidity and mortality in the United States. Thromboembolic events are characterized by an occlusion of a blood vessel. The occlusion can be caused by a clot or thrombus, which can be viscoelastic (jelly-like) and is comprised of platelets, fibrinogen, and other clotting proteins. The occlusion can also be more rigid material such as plaque, which has broken off from a vessel wall upstream of the site of the occlusion.

When a clot occludes an artery, tissue ischemia (lack of oxygen and nutrient delivery to the tissue) can develop. The ischemia can progress to tissue infarction (cell death) if the occlusion persists. Infarction does not develop or is greatly limited if the flow of blood is reestablished rapidly. Failure to re-establish blood flow can lead to the loss of limb, angina pectoris, myocardial infarction, stroke, compromised cognitive or neural function, or even death.

Occlusion of the venous circulation by thrombi leads to blood stasis, which can cause numerous problems. The majority of pulmonary embolisms are caused by emboli that originate in the peripheral venous system. Reestablishing blood flow and removal of the thrombus is important for the well being of the patient. There are many existing techniques employed to reestablish blood flow in an occluded vessel. One common surgical technique, an embolectomy, involves incising a blood vessel and introducing a balloon-tipped device, such as the Fogarty® catheter, to the location of the occlusion. The balloon is then inflated at a point beyond the clot and used to translate the obstructing material back to the point of incision. The surgeon can, then, remove the obstructive material. While such surgical techniques have been useful, exposing a patient to surgery may be traumatic and best avoided when possible. Additionally, the use of a Fogarty® catheter is problematic because of the great risk of damaging the interior lining of the vessel as the catheter is being withdrawn.

Percutaneous methods are also utilized for reestablishing blood flow. A common percutaneous technique is referred to as balloon angioplasty where a balloon-tipped catheter is introduced to a blood vessel, typically through an introducing catheter. The balloon-tipped catheter is then advanced to the point of the occlusion and inflated in order to dilate the stenosis. Balloon angioplasty is appropriate for treating vessel stenosis but is not effective for treating acute thromboemboli. Certain compliant balloons have also been used as temporary neck bridges for coiling cerebrovascular aneurysms with embolic coils or other materials, however the inflated balloons typically block the parent vessel and the patient can only tolerate short-periods, generally inadequate to properly perform embolization of a neurovascular aneurysm, of such ischemic balloon inflation.

Another percutaneous technique is to place a microcatheter near the clot and infuse streptokinase, urokinase, tPA, or other thrombolytic agents to dissolve the clot. Unfortunately, thrombolysis typically takes hours to days to be successful. Additionally, thrombolytic agents can cause severe hemorrhage and in many patients the agents cannot be used at all.

Although neurointerventional devices and procedures have advanced, there remains a need for expeditious restoration of distal flow to blocked, or stenotic, cerebrovascular vessels and for improved devices to treat cerebrovascular aneurysms which, if ruptured, can lead to severe neurological deficit or patient death.

### SUMMARY OF THE INVENTIONS

The present invention provides catheter devices and method for treating disorders in human or animal subjects.

In accordance with the invention, there is provided a catheter device which comprises a proximal shaft member having a proximal end and a distal end; a distal shaft member having a proximal end and a distal end; an expandable member having a distal end connected to the proximal end of the distal shaft member and a proximal end connected to the distal end of the proximal shaft member; and a variable-length member that extends through the expandable member and is transitionable between a) a short configuration having a first axial length and b) a long configuration having a second axial length longer than said first axial length. The expandable member assumes an expanded configuration when the variable-length member is in its short configuration and a contracted configuration when the variable length member is in its short configuration. In some embodiments, the variable-length member may be a curved member that is transitionable between a curved (axially short) configuration and a straight or substantially straight (axially long) configuration.

Further in accordance with the invention, the catheter device may comprise a micro-catheter, having an outside diameter of approximately 3French or smaller, with the incorporation of an outer diametrically expansile/contractile element near the distal region of the device. This expansile/contractile element coupled with a micro-catheter system can serve a variety of therapeutic indications within the cerebrovasculature. Amongst these are occlusive flow restoration, thrombus retrieval, thrombolysis, and temporary neck bridging/neck remodeling of aneurysms. In some embodiments, the micro-catheter can comprise a distention means for vascular anastomotic regions, foreign body retrieval, or an endovascular filter.

In an embodiment, the micro-catheter can comprise means to deliver therapeutic devices and diagnostic agents through one or more of the catheter's lumens or side holes, which further adds to this systems utility. The devices' lumen, or lumens, could allow for aspiration or drainage.

The Multi-Utilitarian Micro-Catheter System can be provided as an axially elongate tubular structure with distal and proximal ends and a lumen throughout its length. The length of the catheter can be approximately 150 cm and can range between 100 cm and 200 cm. The catheter can have an outer diameter with the element contracted of no more than 1 mm (3F).

The outer diametrically expansile/contractile element, hereafter referred to as the expandable element, which can be generally affixed to the catheter shaft near the distal end of the catheter shaft, can be fabricated from a variety of metallic or polymeric materials, either porous, non-porous, or a combination of these materials. This expandable element can be located within the distal region of the design, but preferably about 3-5 cm from the distal tip to improve guidewire aided navigation through tortuous vasculature. The design is provided with the expandable element it's the most expanded configuration, having an outer diameter of 2 mm to 10 mm, but preferably between 2 mm to 7 mm.

To contract the expandable element diametrically, a standard 0.010″ diameter guidewire, or other appropriate size, is introduced with the catheter's lumen and one or more lumen constrictions are provided just distal to the expandable element, with an optional constriction positioned proximal to the expandable element. Once the guidewire is positioned through these constrictions, it provides enough frictionally induced axial force on the distal constriction to cause the expandable element to contract in diameter (and expand the element linearly). The guidewire can also increase the bending stiffness of the catheter system. The proximal constriction is useful in maintaining guidewire position and can be advantageous if the guidewire is not otherwise secured at the proximal end of the catheter system. The distal lumen within the element can be provided with a length of helically disposed tubing, a length of serpentine tubing, a biased coil having a central lumen through which a secondary catheter can be inserted, a telescoping tube set, or a bellows mechanism, which provides a corresponding length alteration of the catheter's lumen to coincide with that of the expandable element. The length of the expandable element can be between 10 mm and 50 mm in the outer diametrically expansile configuration and between 12 mm and 100 mm in length in its contractile, minimum diameter configuration.

Other aspects or embodiments of the inventions include the methods of use. In a first embodiment, the device can be used for the purposes of thrombus engagement, thrombus manipulation, and flow restoration within a partially or totally occluded vessel. In this embodiment, the device is first prepared by flushing, or priming, the lumen with saline. A 0.010″ OD guidewire is then placed within the lumen to contract, inwards or downwards, the outer diameter of the expandable element. The system (catheter and guidewire) are then navigated together to the site of the occlusive thrombus. The catheter and guidewire are advanced through the thrombus so that the expandable element is positioned within the thrombus. Once positioned through the thrombus, the guidewire is then removed (or partially pulled back away for the lumen constrictions). This allows for the element to expand within the thrombus accomplishing two purposes; 1) to entwine the thrombus, pushing it outwardly against the vessel wall, and 2) to allow blood flow restoration to occur to ischemic areas distal of the thrombus. Additionally, diagnostic agents (such as radiographic, MRI, or other contrast agents) can be administered through the catheter lumen to assess the vasculature distal to the occlusive thrombus.

In another embodiment of the methods of use, the catheter can be used to perform targeted thrombolysis. In this embodiment, the device is first prepared by flushing or priming the lumen with saline. A 0.010″ OD guidewire can then be inserted within the lumen to contract, inwards or downwards, the outer diameter of the element. The system (catheter and guidewire) are then navigated together to the site of the occlusive thrombus. The catheter and guidewire are advanced through the thrombus so that the expandable element is positioned within the thrombus. Once the element is expanded, the thrombus is immobilized. Thrombolytic agents, or other therapeutic agents, can be administered directly into the thrombus through side holes located in the wall of the catheter in the region of the expandable element. The side holes operably communicate between the lumen of the catheter and the environment outside the catheter.

In another embodiment of the methods of use, the catheter can be used to perform thrombus retrieval. In this embodiment, the device is first prepared by flushing or priming the lumen with saline. A 0.010″ OD guidewire is then inserted within the lumen to contract, inwards or downwards, the outer diameter of the element. The system is then navigated together to the site of the occlusive thrombus. The catheter and guidewire are advanced through the thrombus so that the expandable element is positioned within the thrombus. The expandable element is expanded, engaging the thrombus. After engaging the thrombus with the expanded element, the user can either administer thrombolytic agents, contract the element by moving forward the guidewire through the constrictions, or both, to further entwine the thrombus. The catheter with entrapped thrombus is then removed from the vasculature. Additionally, the user may elect to keep the element expanded, and remove the catheter device from the vasculature. Lastly, the thrombus removal could be aided by aspiration through the catheter side holes.

In another embodiment of the methods of use, the catheter can be used to perform temporary neck remodeling of aneurysms or other vascular lesions. Often during coil embolization of aneurysms, the aneurismal necks encountered are considered wide, necessitating the need for a neck-bridging device such as a temporary micro-balloon or an implantable stent. These neck-bridging devices hold the coils in place to prevent them from dropping into the parent vessel during delivery. Balloons conform to the inner surface of the vessel wall and provide a smooth surface against the coils, but seal the vessel from blood flow for perhaps long durations, such sealing having potentially catastrophic ischemic consequences if sustained for too long a time. After filling the aneurysm with coils these micro-balloons are deflated and removed for the vasculature. Neurological stents are permanent implants that can bridge the neck during the coiling procedure, they are expensive and non-retrievable, but allow blood flow through them. The design/method concept disclosed herein would be to employ the microcatheter with the expandable element positioned across the neck of the aneurysm and radially expand the element to provide the neck bridge. The element in this case could be provided with a non-porous surface about the cylindrical outer surface portion enabling a smoother, non-open surface against the delivered embolization coils. Other embodiments can comprise a window, a skive, a hole, or a breach in the medial or distal portion of the catheter to allow the introduction of a coil deliver micro-catheter (coaxially) into the aneurysm. In this embodiment, the catheter system may be slightly larger (3Fr-5Fr) than the up to 3Fr diameter typical microcatheter.

In other embodiments, the microcatheter can be used for the purposes of anastomosis distension or dilation, vascular foreign body retrieval, temporary dilatation and flow restoration through atheromatous plaque, and vascular embolic filtering. These goals can be addressed by inserting the proper therapeutic device, such as a dilatation balloon, grasper or basket device, high force mesh dilator, or distal protection filter, respectively, through the working lumen of the microcatheter.

For purposes of summarizing the invention, certain aspects, advantages and novel features of the invention are described herein. It is to be understood that not necessarily all such advantages may be achieved in accordance with any particular embodiment of the invention. Thus, for example, those skilled in the art will recognize that the invention may be embodied or carried out in a manner that achieves one advantage or group of advantages as taught herein without necessarily achieving other advantages as may be taught or suggested herein. These and other objects and advantages of the present invention will be more apparent from the following description taken in conjunction with the accompanying drawings.

### BRIEF DESCRIPTION OF THE DRAWINGS

A general architecture that implements the various features of the invention will now be described with reference to the drawings. The drawings and the associated descriptions are provided to illustrate embodiments of the invention and not to limit the scope of the invention: Throughout the drawings, reference numbers are re-used to indicate correspondence between referenced elements.

FIG. 1A illustrates a side view of a catheter, wherein a guidewire has not yet been inserted into the central catheter lumen, thus the expandable element remains biased in it's fully expanded configuration, according to an embodiment of the invention;

FIG. 1B illustrates a side view of the catheter of FIG. 1A, wherein a guidewire is fully inserted into the catheter lumen resulting in the expandable element being forced into its fully collapsed, minimum diameter configuration, according to an embodiment of the intention;

FIG. 2A illustrates a detail of the distal region of the catheter of FIG. 1A showing the guidewire constriction or aperture and a radially expanded, expandable element, according to an embodiment of the invention;

FIG. 2B illustrates a detail of the distal region of the catheter of FIG. 2A, wherein a guidewire has been inserted through the guidewire constriction forcing the expandable element to contract radially, according to an embodiment of the invention;

FIG. 3A illustrates a thrombus removal catheter in its minimum diameter configuration being advanced toward a mass of thrombus within a blood vessel, according to an embodiment of the invention;

FIG. 3B illustrates the thrombus removal catheter of FIG. 3A, wherein the catheter has been advanced through a central portion of the thrombus such that a radially expandable region extends beyond both ends of the thrombus, according to an embodiment of the invention;

FIG. 3C illustrates the thrombus removal catheter of FIG. 3B, wherein the radially expandable region has been diametrically expanded to contact and entrap the thrombus, according to an embodiment of the invention;

FIG. 4A illustrates the thrombus removal catheter of FIG. 3C, wherein the radially expandable region has been re-collapsed, according to an embodiment of the invention;

FIG. 4B illustrates the thrombus removal catheter of FIG. 4A, wherein the catheter, with entrapped thrombus material, is being withdrawn into a funneled guide catheter, according to an embodiment of the invention;

FIG. 5 illustrates an expandable catheter expanded across a cerebrovascular aneurysm for the purpose of forming a temporary neck bridge, according to an embodiment of the invention, according to an embodiment of the invention;

FIG. 6 illustrates an expandable microcatheter element placed across the entrance to a cerebrovascular aneurysm, wherein the expandable element forms a neck bridge across the opening to the main artery, with an embolic coil being deployed within the aneurysm, according to an embodiment of the invention;

FIG. 7 illustrates an expandable microcatheter element placed across the entrance to a cerebrovascular aneurysm, wherein the expandable element forms a neck bridge across the opening to the main artery, with a quantity of embolic mass being deployed within the aneurysm, according to an embodiment of the invention;

FIG. 8 illustrates the distal end of a microcatheter with an expandable region placed across the entrance to an aneurysm such that a delivery catheter is capable of deploying a coil within the aneurysm, according to an embodiment of the invention;

FIG. 9 illustrates the distal end of a microcatheter with its expandable region dilated within a length of cerebrovasculature, wherein the catheter comprises a serpentine expandable length section within the expandable region, according to an embodiment of the invention;

FIG. 10A illustrates a length of vasculature, partially blocked by a hard plaque formation, being approached by a microcatheter and guidewire, according to an embodiment of the invention;

FIG. 10B illustrates the microcatheter of FIG. 10A having been advanced through the central opening of the plaque, according to an embodiment of the invention;

FIG. 10C illustrates the microcatheter of FIG. 10A and FIG. 10B fully dilated within the region of plaque, thus temporarily relieving the restriction caused by the plaque, according to an embodiment of the invention;

FIG. 11A illustrates a length of vasculature having an aneurysm and a partially dislodged embolic coil projecting into the lumen of the parent vessel, wherein a microcatheter is being advanced toward the dislodged coil, according to an embodiment of the invention;

FIG. 11B illustrates an expandable region of the microcatheter in its fully dilated configuration in the proximity of the aneurysm and the partially dislodged coil, according to an embodiment of the invention;

FIG. 11C illustrates a grasper advanced through the central lumen of the microcatheter, wherein the grasper is snaring an end of the dislodged embolic coil, and further wherein an expandable tip guide catheter has been advanced over the microcatheter to receive the snared coil;

FIG. 12A illustrates a microcatheter being advanced toward an embolic coil which has become partially dislodged from an aneurysm, according to an embodiment of the invention;

FIG. 12B illustrates an expandable member of the microcatheter dilated adjacent to the aneurysm such that the dislodged end of the coil has become entrapped within the mesh of the expandable member, according to an embodiment of the invention;

FIG. 12C illustrates the expandable member having been constricted to a reduced diametric dimension to secure the coil end within its structure, the expandable member being withdrawn proximally into a flared receiving catheter, according to an embodiment of the invention;

FIG. 13A illustrates an expandable member dilated downstream of a thrombus formation through which a microcatheter has been advanced, wherein a membrane partially covers the expandable member, according to an embodiment of the invention; and

FIG. 13B illustrates an expandable member dilated downstream of a thrombus formation wherein a membrane substantially seals the gaps in the entire expandable member, according to an embodiment of the invention.

### DETAILED DESCRIPTION OF THE ILLUSTRATED EMBODIMENTS

The inventions disclosed herein may be embodied in other specific forms without departing from its spirit or essential characteristics. The described embodiments are to be considered in all respects only as illustrative and not restrictive. The scope of the inventions is therefore indicated by the appended claims rather than the foregoing description. All changes that come within the meaning and range of equivalency of the claims are to be embraced within their scope.

As used herein, the terms proximal and distal refer to a direction or a position along a longitudinal axis of a catheter or medical instrument. Proximal refers to the end of the catheter or medical instrument closer to the operator, while distal refers to the end of the catheter or medical instrument closer to the patient. For example, a first point is proximal to a second point if it is closer to the operator end of the catheter or medical instrument than the second point. The measurement term French, abbreviated Fr or F, is defined as three times the diameter of a device, as measured in mm. Thus, a 3 mm diameter catheter is 9 French in diameter.

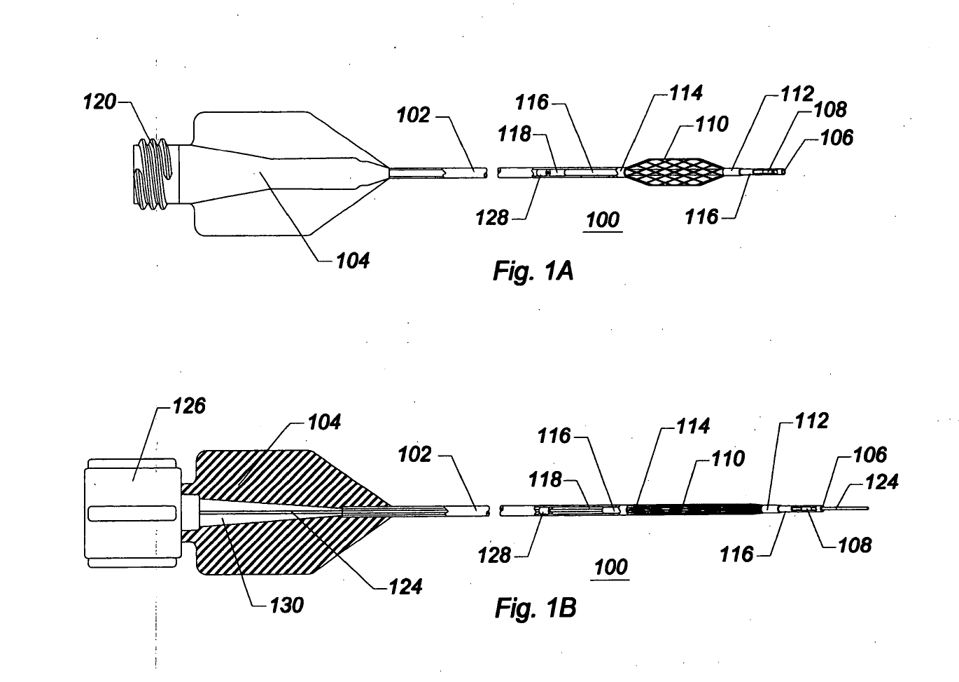

FIG. 1A illustrates a microcatheter 100 comprising an outer shaft 102 further comprising an outer shaft lumen 118, a hub 104 further comprising a proximal Luer lock adapter 120, a distal shaft 116 further comprising a distal shaft lumen 106, a distal constriction 108, a proximal constriction 128, and an expandable member 110 further comprising a proximal bond 114 and a distal bond 112. The expandable member 110 is illustrated in its diametrically expanded configuration.

Referring to FIG. 1A, the proximal end of the outer shaft 102 is affixed to the distal end of the hub 104. The inner lumen 118 of the outer shaft 102 is operably connected to the tapered lumen 130 of the hub 104 such that there are minimal or no bumps or obstructions to passage of catheters or guidewires from the tapered lumen 130 into the outer shaft inner lumen 118. The proximal end of the distal shaft 116 is slidably disposed within the distal end of the outer shaft 102. The proximal end of the expandable member 110 is affixed to the outer shaft 102 by the proximal bond 114. The distal end of the expandable member 110 is affixed to the distal shaft 116 by the distal bond 112. The expandable member 110 retains a minimum and a maximum overall length between the distal bond 112 and the proximal bond 114 such that the overlap distance between the two telescoping tubes 102 and 116 is maintained to a minimum of about 1-cm. The distal constriction 108 is affixed within the lumen 106 to the distal shaft 116. The distal constriction 108 further comprises a central lumen (not shown) having an undeformed, or unstressed, diameter smaller than that of the guidewire 124 meant to be inserted therethrough. The lumen diameter of the distal constriction 108, or region of reduced diameter, can have a diameter of between 100% and 10% of the guidewire 124, and preferably between 40% and 80% of that of the guidewire 124. The central lumen (not shown) of the distal constriction 108 can expand to accommodate insertion of the guidewire 124 but imparts substantial friction on the guidewire 124. The proximal constriction 128 is affixed to the interior wall of the outer shaft 102 and within the lumen 118. The proximal face of the proximal constriction 128 can have an inwardly tapered funnel-like lead-in to the central lumen of the proximal constriction 128. This lead-in (not shown) can facilitate coercing the guidewire 124 into the central lumen of the proximal constriction 128. This is especially important in the larger diameter inner lumen 118 of the outer shaft 102. The guidewire 124 can be an elongate member configured to collapse/expand the expandable member or region 110. The elongate member may also be a linkage, mechanical linkage, pushrod, push-pull rod, or the like. The guidewire 124, or linkage, preferably comprises the properties of high column strength, high tensile strength, low elongation and high flexibility.

The hub 104 can be affixed to the outer shaft 102 by processes such as, but not limited to, adhesive bonding, heat welding, overmolding, insert-molding, ultrasonically welding, or the like. The proximal bond 114 and the distal bond 112 can be created using processes such as, but not limited to, adhesive bonding, heat welding, overmolding, insert-molding, ultrasonic welding, wrapping, mechanical fixation, encapsulation, and the like.

The overall working length of the microcatheter 100 can range between 50 cm and 200 cm with a preferred range of 100 cm to 175 cm. The outside diameter of the outer shaft 102 can range between 0.5 French and 10 French with a preferred range of 1 French and 4 French. The length of the expandable member 110 in its radially expanded configuration can range between 1 cm and 20 cm with a preferred length range of 2 cm and 10 cm and a most preferred range of 2.5 cm to 5 cm. The length of the tapered regions at the end of the expandable member 110 can each range between 5% and 40% of the total length of the expandable member 110. The expandable member 110 can have an expanded diameter ranging from 1 French to 13 French with a preferred diameter of 2-French to 5 French. The diameter of the guidewire 124 can range between 0.005 and 0.015 with a preferred range of 0.008 to 0.012.

The materials appropriate to the construction of the microcatheter 100 are biocompatible and sterilizable. The outer shaft 102 and the distal shaft 116 can be fabricated from relatively materials such as, but not limited to, PTFE, Pebax, Hytrel, polyurethane, polyethylene, polyimide, polyamide, polyester, PEEK, and the like. The construction of the distal shaft 116 and the outer shaft 102 can be such that flexibility, torqueability, and column strength, all beneficial to a catheter, are maintained. The distal shaft 116 and the outer shaft 102 can be of singular material construction or one or both can be of composite, or built-up, construction. Such composite construction can comprise a polymeric inner and outer coat or surround enveloping a reinforcement layer. The reinforcement layer can comprise braid, coil, or stent-shaped construction fabricated from materials such as, but not limited to, stainless steel, tantalum, titanium, nitinol, polyester, PEN, cobalt nickel alloy, polyamide, polyimide, and the like. The hub 104 can be fabricated from more rigid materials such as, but not limited to, acrylonitrile butadiene styrene (ABS), polyethylene, polypropylene, polyamide, polyimide, polyether ether ketone (PEEK), polysulfone, and the like. The mesh 110 can be fabricated from nitinol, stainless steel, titanium, cobalt nickel alloy, tantalum, polyimide, polyamide, polyester, and the like. In other embodiments, the outer shaft 102 can have variable flexibility characteristics along its length. In certain embodiments, the outer shaft 102 can comprise continuously varying properties. In certain of the continuously varying property embodiments, the outer shaft 102 can be progressively more flexible moving from the proximal end toward the distal end. In certain embodiments, the outer shaft 102 can comprise a plurality of regions of discreet flexibility. The number of regions of discreet flexibility can range between 2 and 10 and preferably between 2 and 5. The regions closer to the distal end can be made advantageously more flexible than regions closer to the proximal end of the outer shaft 102. Such changes in flexibility, for example moving from higher stiffness to lower stiffness, can be achieved by methods such as, but not limited to, changing the polymer composition to lower hardness materials, changing the pitch of a coil reinforcement to provide greater spacing between coils, changing the pitch of a braided reinforcement to achieve greater spacing, changing the thickness of the wires used in a coil or braid to smaller dimensions, or the like.

The bars of the mesh 110 can comprise round, oval, rectangular, or other suitable cross-sectional shape. The mesh 110 can also be configured as a slotted tube, or a plurality of bars oriented substantially parallel to the longitudinal axis of the distal member 116. The mesh 110 can also be configured with all the patterns disclosed for various implantable stent devices.

The overlap region between the distal shaft 116 and the outer shaft 102 permits relative motion between the two shafts 116 and 102 while the expandable member 110 changes its length in response to operator control. This length changing feature can also be accomplished by affixing a helically disposed tube, a serpentine tube, a coil, a braided tube, or other structure that can substantially maintain its shape but change length in response to external forces to the outer shaft 102, the distal shaft, 116, or both.

FIG. 1B illustrates the microcatheter 100 of FIG. 1A illustrated with a guidewire 124 inserted therethrough and the expandable member 110 in its diametrically constricted configuration. The microcatheter 100 comprises the hub 104, which is illustrated in cross-section, and further comprises a tapered lead in lumen 130. The microcatheter 100 further comprises the outer shaft 102, the outer shaft lumen 118, the distal shaft 116, the distal shaft lumen 106, the distal constriction 108, the proximal constriction, the distal bond 112, the proximal bond 114, the guidewire 124, and the guidewire proximal cap 126.

Referring to FIG. 1B, the guidewire 124 has been inserted through the lumen 130 of the hub 104 and into the lumen 118 of the outer shaft 102. The guidewire 124 is routed through the optional proximal constriction 128, into the inner lumen 106 of the distal shaft 116, through the distal constriction 108 and out the distal end of the inner lumen 106. It is also possible that the guidewire 124 will not pass entirely through the distal constriction 108, in which case, the guidewire 124 would also not extend beyond the distal end of the inner lumen 106. The guidewire proximal cap 126 is affixed to the proximal end of the guidewire 124. The guidewire proximal cap 126 is removably affixed to the Luer lock fitting 120 on the hub 104. The guidewire 124 is longitudinally fixed relative to the inner shaft 118 and the distal constriction 108 by locking the cap 126 to the hub 104. Distal motion of the guidewire 124 through the distal constriction 108 causes sufficient longitudinal stretching force, due to application of friction by the distal constriction 108, such that the expandable member 110 becomes stretched longitudinally to its maximum extent and thus the expandable member 110 assumes its minimum radial dimension.

The proximal constriction 128 is optional but the friction supplied by the proximal constriction 128 on the guidewire 124 can be used to stabilize the guidewire and maintain the expandable member 110 in its fully stretched state without the need for the cap 126. Note that the distal constriction 108 and the proximal constriction 128 are of different outside diameters to permit them to be affixed inside different diameter tubes but the diameters of the constrictions 108 and 128 can be tailored to the specific configuration of the catheter. The constrictions 108 and 128 can be of single material or multiple material layer construction. They can be fabricated from materials configured to generate high friction such as, but not limited to, silicone elastomer, latex rubber, thermoplastic elastomer, polyurethane, and the like. These elastomeric materials can be fabricated free from oils or other lubricants and with surface properties that generate high friction on the outside surface of the guidewire 124. The guidewire 124 can beneficially be constructed using an outer surface that is non-lubricious. Thus the guidewire 124 can have at least a part of its outer surface free from coating with materials such as PTFE, Teflon, FEP, or the like.

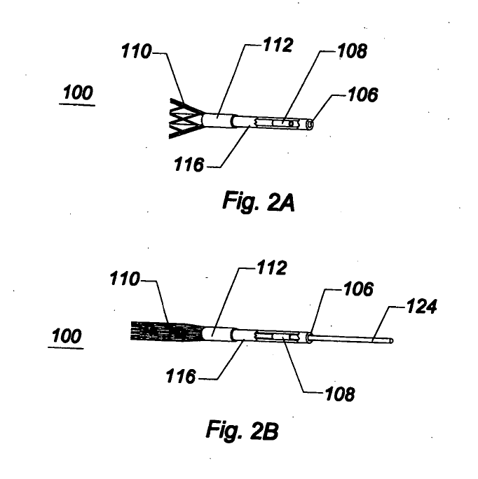

FIG. 2A illustrates a detailed image of the distal end of the microcatheter 100, with the expandable member 110 in its diametrically expanded state, further comprising the distal bond 112, the distal shaft 116 further comprising the lumen 106, and the distal constriction 108.

Referring to FIG. 2A, the expandable member 110, in the illustrated embodiment, is a mesh or braid of filaments, the mesh being bonded to the distal shaft 116 by the distal bond 112. The distal shaft 116 is shown in partial breakaway view to reveal the distal constriction 108. The mesh expandable member can be malleable, elastomeric or configured as a spring, or it can be shape memory. The mesh 110, in its malleable configuration can be fabricated from annealed stainless steel, tantalum, gold, platinum, platinum-iridium, titanium, annealed cobalt nickel alloy, certain aforementioned polymers, and the like. In an embodiment where the mesh 110 is elastomeric, the fibers or filaments can be fabricated from materials such as, but not limited to, spring hardness stainless steel, cobalt nickel alloy, superelastic nitinol, shape memory nitinol, or the like. In certain elastomeric embodiments, the expandable member or mesh 110 can be biased into its maximum diameter configuration so that upon removal of any stretching force, the expandable member 110 assumes its maximum diameter configuration. In the case of nitinol, an austenite finish (Af) temperature of 20° C. or lower, and preferably 15° C. or lower, is beneficial to maximize spring properties at body temperature. In embodiments where the expandable member 110 comprises shape memory properties, the expandable member 110 can be fabricated from nitinol and have an austenite finish temperature of around 28 to 32° C. in order to permit full expansion radially at about body temperature of around 37° C. In the case of nitinol embodiments, either superelastic, pseudoelastic, or shape memory, the nitinol structure can be shape set into the desired configuration to which it will remain biased, near or above its austenite finish temperature. In the illustrated embodiments, removal of any external forces can include removing the guidewire 124 from within the lumen 106 of the distal shaft 116.

FIG. 2B illustrates a detailed image of the distal end of the microcatheter 100, with the expandable member 110 in its diametrically compressed, longitudinally expanded state. The microcatheter 100 further comprises the distal bond 112, the distal shaft 116 further comprising the inner lumen 106, the distal constriction 108, and the guidewire 124.

Referring to FIG. 2B, the guidewire 124 is inserted through the constriction 108, the frictional interference of which forces the distal shaft 116 to move distally to the extent possible and stretching the expandable member 110 to the extent possible. The distal shaft 116 is shown in partial breakaway view to reveal the distal constriction 108. The individual fibers of the expandable member 110 can be seen in their longitudinally expanded configuration with the fibers being oriented more axially or longitudinally than in FIG. 2A.

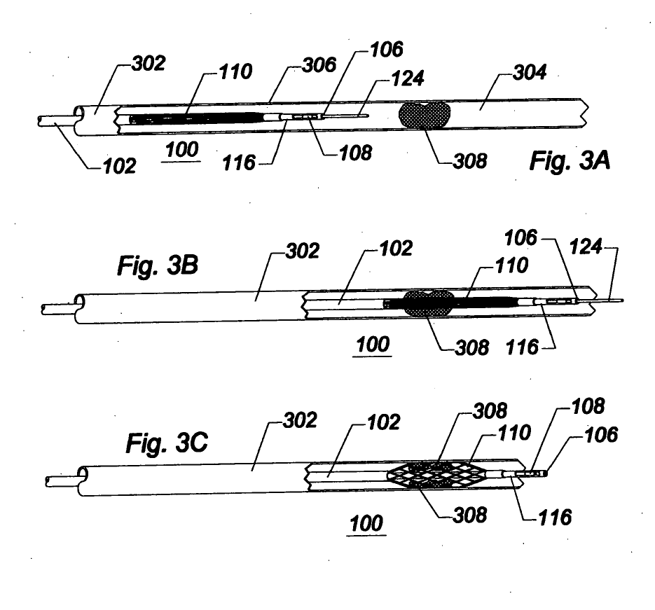

FIG. 3A illustrates a length of blood vessel 302 comprising a lumen 304 and a wall 306. A microcatheter 100 has been inserted into the lumen 304 and is being advanced toward a thrombus or thrombotic mass 308, which is the target of the procedure. The microcatheter 100 comprises the outer shaft 102, the expandable region 110, the distal shaft 116, the inner shaft lumen 106, the distal constriction 108, and the guidewire 124. The microcatheter 100 can be advanced through a guide catheter (not shown), which serves as a tracking device to maneuver the microcatheter 100 toward the therapeutic or diagnostic target 308.

FIG. 3B illustrates the blood vessel 302 wherein the microcatheter 100 has been advanced through the target thrombus 308 and the thrombus 308 is positioned over the expandable region 110. The microcatheter 100 further comprises the outer shaft 102, the distal shaft 116, the inner shaft lumen 106, and the guidewire 124. The blood vessel 302 is shown in partial breakaway view.

FIG. 3C illustrates the blood vessel 302 wherein the expandable region 110 has been dilated to its maximum diameter within the thrombus 308. Referring to FIGS. 3B and 3C, the diametric expansion of the expandable region 110 was performed by removing the guidewire 124 from the microcatheter 100. The expandable region 110, a spring biased mesh, has self-expanded. Additional expansion could be generated by not fully withdrawing the guidewire 124 but applying proximal force on the distal shaft 116 by friction coupling between the guidewire 124 and the distal constriction 108 of FIG. 3A.

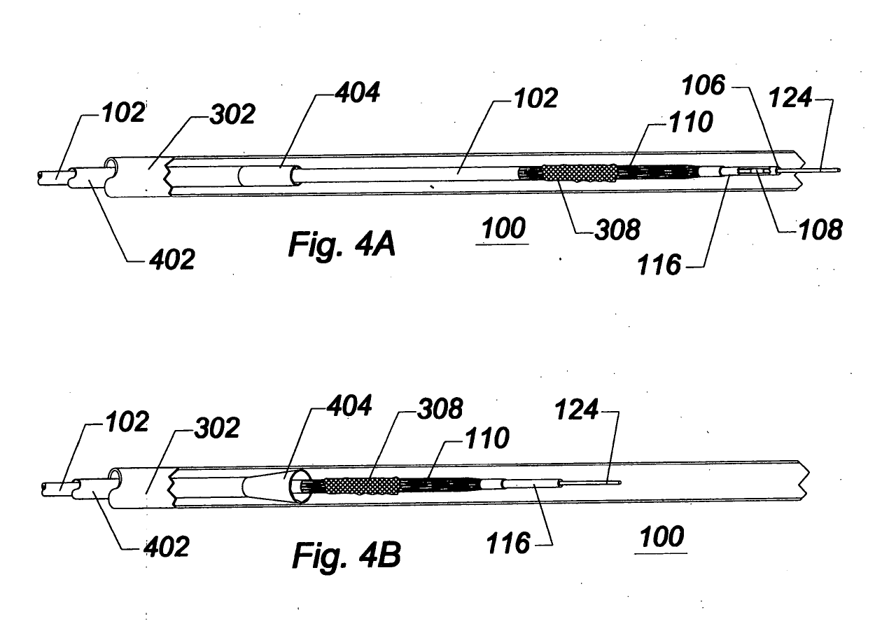

FIG. 4A illustrates the blood vessel 302 with the microcatheter 100 advanced within the thrombotic mass 308 and the expandable region 110 having been re-collapsed by re-insertion of the guidewire 124 through the distal constriction 108. A guide catheter 402 has been advanced distally over the outer shaft 102. The guide catheter 402 further comprises a distal, adjustable flaring region 404, which is affixed to the distal end of the tubing of the guide catheter 402.

FIG. 4B illustrates the blood vessel 302 with the microcatheter 100, further comprising the outer shaft 102 and the expandable region 110, being withdrawn proximally and taking with it the thrombotic mass 308, which has become entwined within the expandable region 110. The adjustable flaring region 404 has been expanded at its distal end to coerce, at least partially, the thrombus 308 and the expandable region 110 inside the guide catheter 402. The guidewire 124 remains in place within the distal shaft 116 to maintain the stretched configuration of the expandable region 110. Once inside the guide catheter 402, the thrombus 308 can be constrained and remnants thereof can be prevented from flaking off and flowing back through the vasculature when the guide catheter 402 and the microcatheter 100 are being removed from the vasculature.

Referring to FIG. 4B, the construction of the guide catheter 402 can be the same as, or similar to, that of the microcatheter 100. The distal flaring region 404 can comprise radially expandable elements that can be activated using shape-memory properties. The shape-memory properties can be activated using body temperature or Ohmic heating to temperatures above that of body temperature. Upon removal of the higher temperatures, in the case of the Ohmic or resistive heating embodiments, the distal flaring region 404 can be made to assume a martensitic, or soft, characteristic conducive to removal of the guide catheter 402 from the vasculature 302. Such elevated temperatures can be generated by electrical current applied across electrical leads that run from the proximal end of the guide catheter 402 to the distal end where they are connected to each end of a nitinol expandable structure or to high-resistance wires such as those fabricated from nickel-chromium metal. The high-resistance wires can be formed along, around, or through the nitinol structure to provide optimum heat transfer to the nitinol. The electrical energy can be applied at the proximal end of the guide catheter 402 by the operator using batteries, or other electrical power supply.

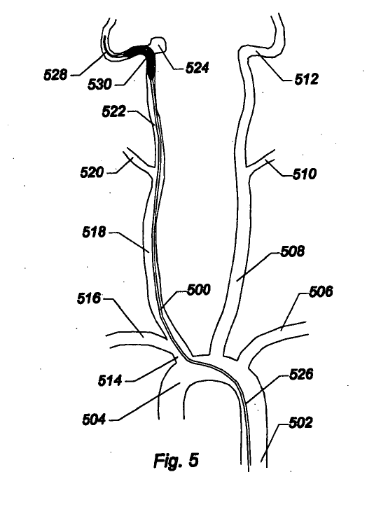

FIG. 5 illustrates a cranial portion of a human circulatory system comprising a descending aorta 502, an aortic arch 504, a left subclavian artery 506, a right subclavian artery 516, an innominate artery 514, a left common carotid artery 508, a right common carotid artery 518, a left external carotid artery 510, a right external carotid artery 520, a left internal carotid artery 512, a right internal carotid artery 522, a cerebrovascular aneurysm 524, a temporary neck bridge microcatheter 500, further comprising a catheter shaft 526, an expandable neck bridge region 530, and a guidewire 528.

Referring to FIG. 5, the microcatheter 500 has been routed from a femoral percutaneous insertion site (not shown) through the femoral and iliac arteries (not shown), and into the aorta 502, where it is next advanced through the innominate artery 514 and into the common carotid artery 518 and finally through the internal carotid artery 522 past the aneurysm 524 target site. The microcatheter 500 can have been routed through a guide catheter (not shown), placed during an earlier step in the procedure. The primary purpose of the guidewire 528 is to control the expansion and contraction of the neck bridge expandable region 530, although it could also be used to assist with guiding the microcatheter 500 to the target site. With the guide wire 528 removed, a separate embolic material delivery catheter (not shown) can be advanced through the guidewire lumen of the microcatheter 100 and be directed through the expandable neck bridge 530 into the aneurysm 524.

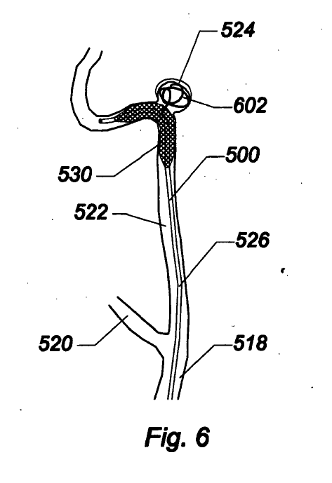

FIG. 6 illustrates a portion of the left human carotid artery tree comprising the common carotid artery 518, the external carotid artery 520, the internal carotid artery 522, and an aneurysm 524. A microcatheter 500, comprising a catheter shaft 526 and an expandable mesh 530, has been advanced toward the aneurysm 524 and a mesh 530 has been expanded across the neck of the aneurysm 524 to form a neck bridge having porosity to blood and small diameter devices. An embolic coil 602 is being deployed within the sac of the aneurysm 524 as part of an embolization procedure.

Referring to FIG. 6, the expandable mesh 530 is capable of forming a porous barrier across the neck of the aneurysm while maintaining blood flow within the parent internal carotid artery 522. The expandable mesh 530 comprises openings between the mesh elements or strands and the openings are capable of passing small catheters, pushers, delivery devices, and the like (e.g., therapeutic or diagnostic instruments) which can be directed to the aneurysm 524 for therapeutic or diagnostic purposes. The guidewire 528, illustrated in FIG. 5 has been removed and replaced with the delivery system for the embolic coil 602. The catheter shaft 526 and the expandable mesh 530 are flexible and capable of bending around tortuous anatomy as is often found in the cerebrovasculature.

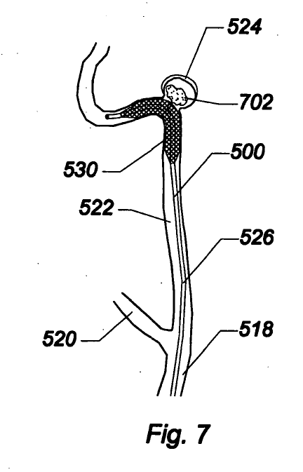

FIG. 7 illustrates a portion of the left human carotid artery tree comprising the common carotid artery 518, the external carotid artery 520, the internal carotid artery 522, and an aneurysm 524. A microcatheter 500, comprising a catheter shaft 526 and an expandable mesh 530, has been advanced toward the aneurysm 524 and a mesh 530 has been expanded across the neck of the aneurysm 524 to form a neck bridge having porosity to blood and small diameter devices. A volume of embolic material 702 is being deployed within the sac of the aneurysm 524 as part of an embolization procedure.

Referring to FIG. 7, the embolic material 702 is being delivered through a liquid delivery catheter routed through the central lumen of the catheter shaft 526 following removal of any guidewires 528 such as those illustrated in FIG. 5. The embolic material 702 is preferably liquid or a very thin gel to permit injection through the liquid delivery catheter. The embolic material 702 can comprise polymers dissolved within solvents such as DMSO or the like, wherein upon exposure to the body environment, the DMSO or other solvent is absorbed by body tissues leaving the polymeric mass to harden into a rigid or semi-rigid embolic structure. Other embolic materials can comprise cyanoacrylate adhesives, tantalum powder, and other additives such as polymeric agents, for example. The embolic material 702 can be used alone or in conjunction with the coils 602 illustrated in FIG. 6. The catheter 500 used for this procedure, as illustrated in FIG. 7, need not be substantially different from the catheter 500 used in the procedure shown in FIG. 6.

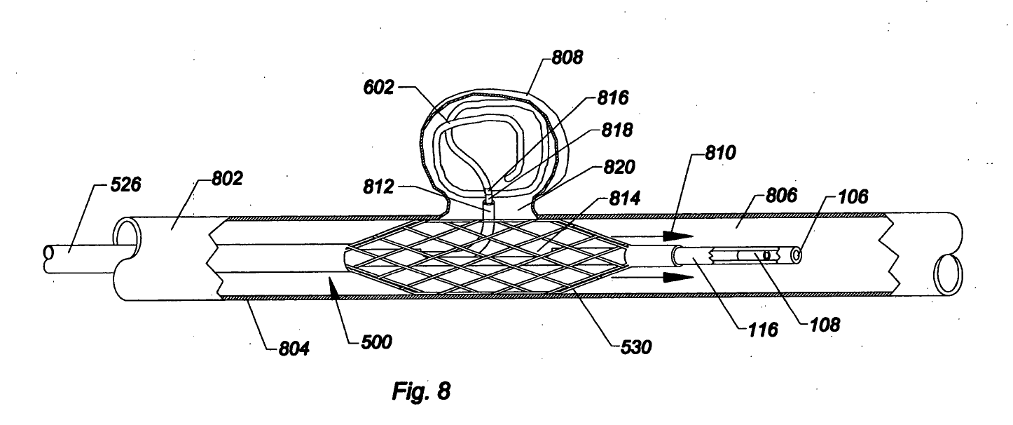

FIG. 8 illustrates a more detailed view of the distal end of a microcatheter 500 configured as a temporary neck bridge for an aneurysm. The microcatheter 500 comprises the mesh 530, the primary shaft 526, the secondary or distal shaft 116 further comprising a lumen 106, a distal constriction 108, a side window 814, a coil delivery catheter 812, a coil pusher 818, a coupler 816, and the embolic coil 602. The microcatheter 500 is illustrated deployed within a blood vessel 802 further comprising a wall 804, a lumen 806, an aneurysm 808 further comprising an aneurysm neck 820, and a volume of flowing blood 810.

Referring to FIG. 8, the guidewire 528 of FIG. 5 is not illustrated because it is removed to create room for the coil delivery catheter 812 and because its withdrawal from the distal constriction 108 permits recovery of the mesh 530 to its fully expanded configuration. The coil delivery catheter 812, or pusher, can further comprise a releasable coupler 816 at its distal end that controllably and reversibly joins the embolic coil 602 to the coil delivery catheter 812. The coil delivery catheter 812 is configured with a distal arc, or bend, so that upon exposure to the side window 814, the catheter 812 curves out of the window toward the aneurysm into which it can now be advanced. The catheter 812 is smaller in diameter than the openings in the mesh 530 to permit passage through the mesh filaments. The coil delivery catheter 812 can be a guide for a pusher 818, as illustrated, or it can, itself, be the coil pusher 818. The coupler 816 can operate due to erosion of a fusible link, release of a mechanical interlock, release of a friction bond, electrolytic detachment, or the like.

The application of the microcatheter 500 as a porous neck bridge permits partial closure of the neck 820 of the aneurysm 808, thus reducing flow washout effects that could dislodge embolic material. The expandable mesh 530 is porous and permits blood to flow through the mesh 530 following diametric expansion, thus maintaining distal perfusion. This is a superior technique to the prior art that involves total blockage of the neck 820 of the aneurysm 808 and parent vessel lumen 806 with a balloon during embolic material delivery. Such prior art total blockage can last for periods of time in excess of those tolerable to cerebral tissues. Eliminating cerebral tissue ischemia facilitates better patient outcomes following procedures where placement of a temporary neck bridge across an aneurysm 808 is indicated. Increasing the time of temporary neck bridge placement eases the burden on the interventional neuroradiologist and permits more accurate therapeutic procedures with superior patient outcomes. The microcatheter 500 can be configured to reach into the vasculature as far as the carotid siphon with an outside diameter of around 2 to 4 French. The microcatheter can be configured to reach into the cerebrovasculature as far as the Circle of Willis and beyond into the middle cerebral artery as far as the M1 bifurcation with a diameter of 1 to 3 French. The size of corresponding catheter components can be scaled appropriately to the catheter outside diameter.

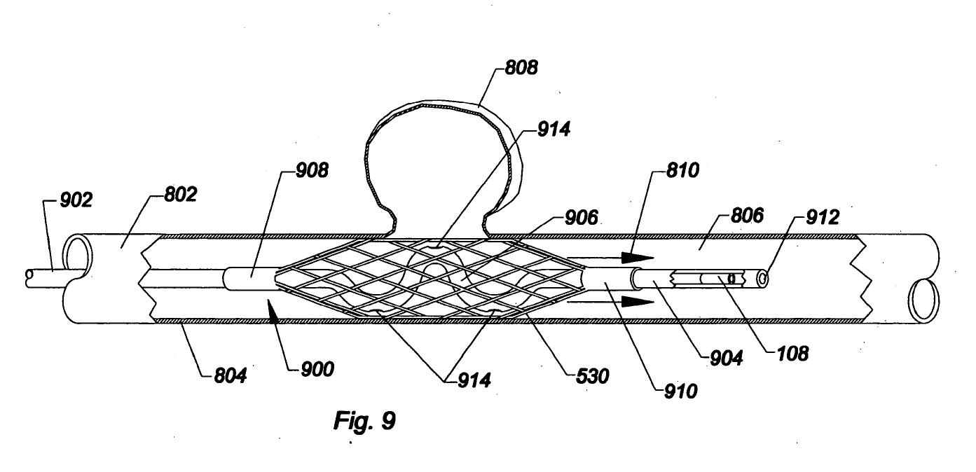

FIG. 9 illustrates an embodiment of the microcatheter 900 comprising a proximal shaft 902, a distal shaft 904, a serpentine adjustable length shaft 906, and an expandable region 530. The serpentine adjustable length shaft 906 further comprises a plurality of fenestrations ports, or holes 914. The distal shaft 904 further comprises a central lumen 912 and a constriction 108. The expandable region 530 further comprises a proximal bond 908 and a distal bond 910. The microcatheter 900 is illustrated being advanced inside a blood vessel 802 comprising a wall 804, a lumen 806, an aneurysm 808, and a volume of flowing blood 810 within the lumen 806.

Referring to FIG. 9, the expandable region 530 is being used as a temporary neck bridge to create a porous barrier across the neck of the aneurysm 808. The expandable region 530 is bonded to the proximal shaft 902 by the proximal bond 908 and to the distal shaft 904 by the distal bond 901. The serpentine adjustable length shaft 906 is bonded, welded, integral to, or otherwise affixed to the proximal shaft 902 and the distal shaft 904. The constriction 108 is affixed to the walls of the interior lumen 912 of the distal shaft 904. The holes 914 are integral to the wall of the serpentine adjustable length shaft 906. The holes 914 operably connect the interior lumen (not shown) of the serpentine adjustable length shaft 906 to the exterior environment around the outside of the shaft 906, the environment being substantially within the volume encompassed by the expandable region 530.

The expandable region 530 can comprise a mesh, as illustrated, or it can comprise a plurality of longitudinal bars or struts spaced circumferentially around the axis of the microcatheter 900. The expandable region 530 can, in other embodiments, comprise mesh structures at the proximal end, distal end, or both, and interconnecting longitudinal struts between the mesh proximal and distal ends. The serpentine adjustable length shaft 906 can comprise polymeric materials or polymeric layered construction with a central reinforcement. The polymeric materials used in the serpentine adjustable length shaft 906 can, in some embodiments, comprise elastomeric materials to permit the shaft 906 to assume a bias toward a pre-set configuration. The pre-set configuration can comprise a coil configuration or an undulating or wavy configuration. The pre-set configuration can be fabricated by methodologies such as heat-setting, casting the tube over a spiral mandrel, etc. The shaft 906 is configured such that it can straighten out either by having its ends be placed in tension, as with a guidewire pushing on the constriction 108, by a substantially straight catheter (not shown) being inserted therethrough, or both. In a preferred embodiment, the expandable region 530 is in its radially collapsed configuration when the serpentine shaft 906 is in its straightened configuration.

The holes 914 can be used for infusion of thrombolytic agents such as, but not limited to, urokinase, streptokinase, tissue plasminogen activator (tPA), or the like. In other embodiments, the holes 914 can also be used to infuse thrombogenic or embolic materials into an aneurysm 808, for example, or for infusion of dye contrast agents for radiographic purposes.

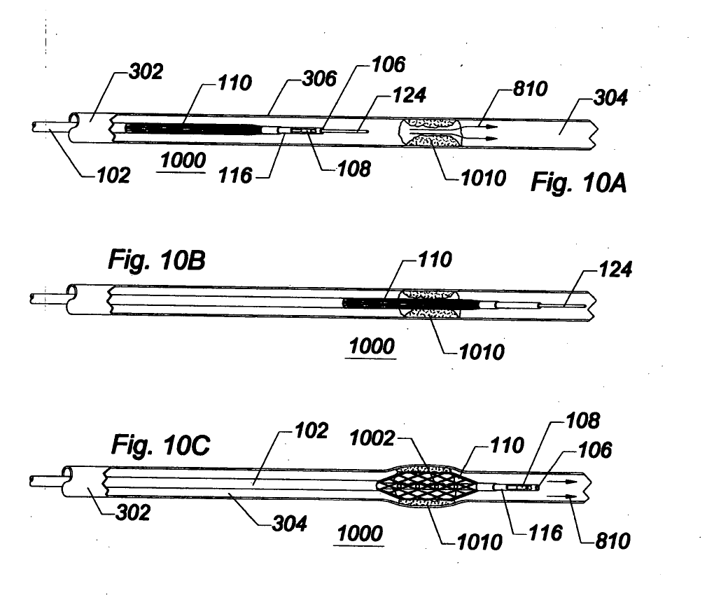

FIG. 10A illustrates a microcatheter 100 being advanced, over a guidewire 124, toward a partially occluding thrombus 1010 adherent to the interior wall 306 of the blood vessel 302. The thrombus 1010 partially occludes the lumen 304 causing stenosis of the blood flow 810. The microcatheter 100 further comprises the proximal shaft 102, the distal shaft 116, the constriction 108, the lumen 106 of the distal shaft 116, and the expandable region 110.

Referring still to FIG. 10A, the expandable region 110 is collapsed to approximately its minimum lateral profile by the distal force exerted by the guidewire 124 against the constriction 108. The microcatheter 100 is being advanced, in some embodiments, using fluoroscopic monitoring and guidance with the aid of radiopaque markers strategically affixed to the microcatheter 100.

FIG. 10B illustrates the microcatheter 100 having been advanced through the thrombus region 1010 with the radially collapsed expandable region 110 placed approximately across the thrombus region 1010. The guidewire 124 is illustrated still in place within the microcatheter 100 to prevent diametric expansion of the expandable region 110.

FIG. 10C illustrates the microcatheter 100 with its expandable region 110 having been expanded by removal of the guidewire 124 (refer to FIG. 10B). The microcatheter 100 further comprises the proximal shaft 102, the distal shaft 116 further comprising the lumen 106 and the plurality of side holes 1002, and the constriction 108 being free from force since the guidewire is removed. The vessel 302 continues to support blood flow 810 within its lumen since the expandable region is porous to the flow of blood, due to the large fenestrations between the mesh elements. The thrombus 1010 is expanded radially outward to provide a central flow region within the vessel 302 that is free from clinically relevant obstruction. In some embodiments, holes or openings 1002 in the wall of the distal shaft 116, disposed beneath the expandable region 110, can be used for the infusion of thrombolytic agents described in FIG. 9. During infusion of the thrombolytic agents, a distal plug (not shown), located near the constriction 108 can prevent escape of the thrombolytic agents out the distal end of the lumen 106. In another embodiment, the guidewire 124 can be configured with a diameter small enough to permit annular flow thereby, but plug or close the hole in the constriction 108 to prevent substantial loss of agent through the distal end. The embodiments described herein are especially suited to rapid treatment of occlusive or ischemic stroke.

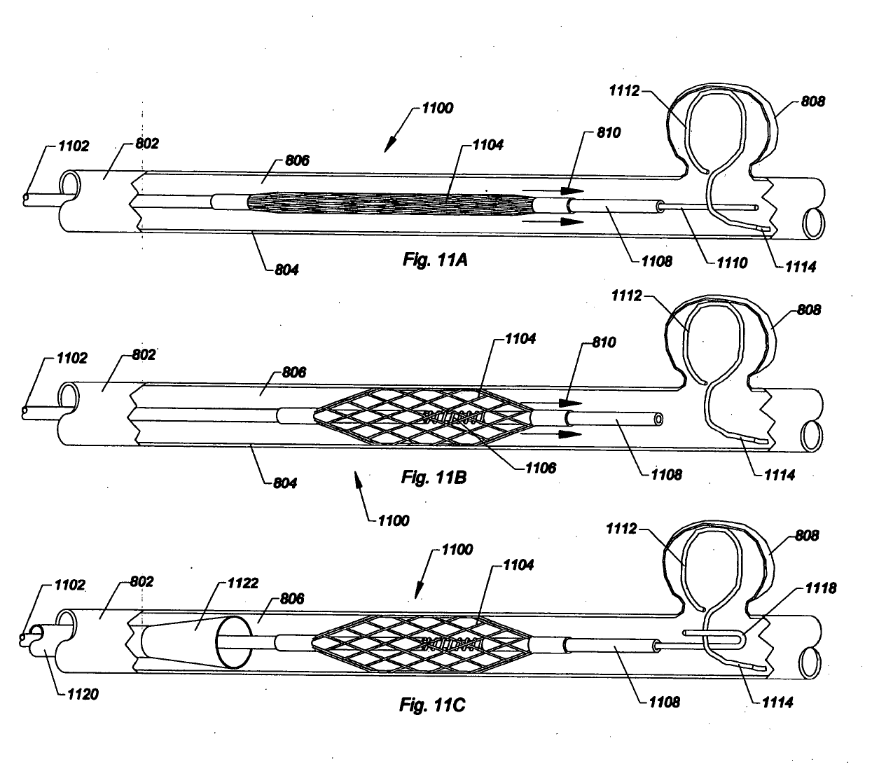

FIG. 11A illustrates a microcatheter 1100 being advanced toward an aneurysm 808 in a vessel 802. The vessel 802 further comprises a vessel wall 804, a vessel lumen 806, and a volume of flowing blood 810. The microcatheter 1100 further comprises a proximal shaft 1102, a distal shaft 1108, an expandable region 1104, and a guidewire 1110, which is shown inserted through the central lumen and which maintains the diametrically collapsed configuration of the expandable region 1104. An embolic coil 1112 is illustrated partially lodged within the aneurysm 808 with the proximal section 1114 of the coil 1112 having escaped into the lumen 806 of the parent vessel 802. The expandable region 1104 is illustrated in its diametrically collapsed configuration. In the illustrated embodiment, the expandable region 1104 is a mesh structure. The proximal tail or section 1114 could generate thrombus, thromboemboli, or itself become fully dislodged and float downstream to embolize the lumen 806 of the parent vessel 802.

FIG. 11B illustrates the microcatheter 1100 with its expandable region 1104 having been fully expanded radially in response to removal of the guidewire 1110. The microcatheter 1100 comprises the proximal shaft 1102, the distal shaft 1108, the expandable region 1104, which is a mesh in the illustrated embodiment, and a coil length adjusting region 1106. The vessel 802 comprises the wall 804, the lumen 806, the aneurysm 808, and the volume of flowing blood 810. The proximal tail 1114 of the embolic coil 1112 continues to protrude into the lumen 806.

FIG. 11C illustrates the microcatheter 1110 with a snare 1118 inserted through the central lumen of the microcatheter 1110. A guide catheter 1120 has been advanced over the proximal shaft 1102, the guide catheter 1120 further comprising a controllably, or selectively, flared distal end 1122. The snare 1118 has hooked the proximal tail 1114 of the coil 1112 in preparation for proximal retraction into the flared guide catheter 1120 and ultimate removal of the coil 1112 from the lumen 806 of the parent vessel 802.

Referring to FIG. 11C, the flared distal end 1122 is affixed to the distal end of the guide catheter 1120. The flared distal end 1122 can be an expandable structure configured with a braid or plurality of longitudinal, bendable elements, and a pull-wire (not shown) which can be used to axially contract the braid, resulting in radial expansion. Alternatively, in other embodiments, the flared distal end 1122 can comprise nitinol shape-memory elements that expand in response to applied electrical current and subsequent resistive heating, or it can expand in response to exposure to blood at body temperature. In yet another embodiment, the flared distal end 1122 can be made to expand in response to removal of a sheath, shroud, or jacket restraint.

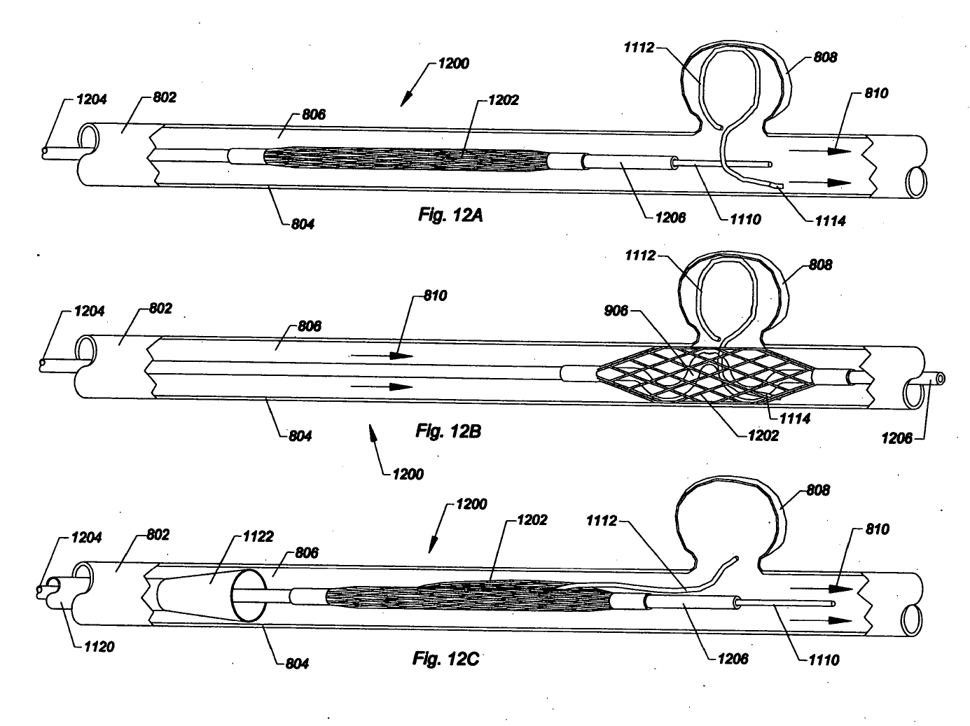

FIG. 12A illustrates a microcatheter 1200 being advanced toward a partially dislodged tail or end 1114 of an embolic coil 1112. The coil 1112 is placed in an aneurysm 808 in the wall 804 of a parent vessel 802, further comprising a lumen 806 and filled with a volume of flowing blood 810. The microcatheter 1200 comprises a proximal shaft 1204, a distal shaft 1206, an expandable region 1202, and a guidewire 1110.

FIG. 12B illustrates the microcatheter 1200 with the guidewire 1110 removed and the expandable region 1202 in a diametrically expanded configuration. The tail 1114 is trapped within the expanded mesh of the expandable region 1202. In the illustrated embodiment, the expandable region 1202 is a mesh. However, the expandable region 1202 can also be configured as a plurality of longitudinal bars, a serpentine stent-like structure, a slotted tube, a wire basket, or the like. The microcatheter 1200 comprises a serpentine length-adjustable element 906 as described in the text accompanying FIG. 9.

FIG. 12C illustrates the microcatheter 1200 with the expandable region 1202 in its diametrically collapsed or minimum profile configuration. The guidewire 810 has been inserted to straighten the length changing region 906, engaging a constriction (not shown), or both, thus forcing the axial length increase and diametric decrease in the mesh 1202. The tail 1114 of the coil 1112 is trapped within the expandable region 1202 and is in the process of being withdrawn from the aneurysm 808. A guide catheter 1120 with a flared distal end 1122 has been advanced over the proximal shaft 1120 to assist with recovery of the misplaced embolic coil 1112.

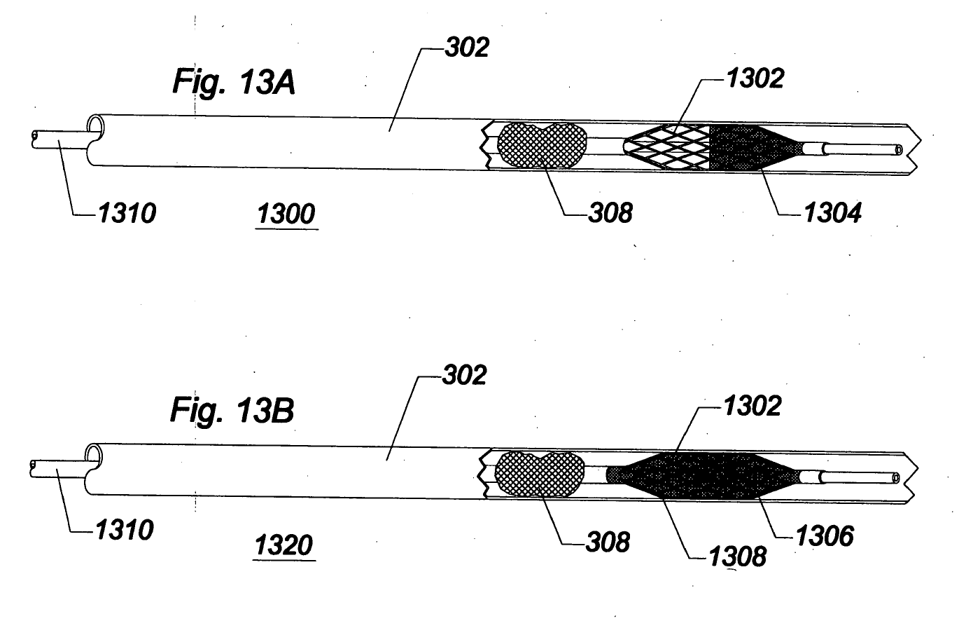

FIG. 13A illustrates a body vessel 302 with an obstruction 308 disposed therein. A microcatheter 1300 has been advanced through the obstruction 308 and an expandable member 1302 has been expanded diametrically. The microcatheter 1300 further comprises a proximal shaft 1310 and a distal expandable member cover 1304.

Referring to FIG. 13A, the expandable member 1302 comprises a mesh, braid, plurality of longitudinal filaments, or the like. The expandable member 1302 is covered, on its exterior, by the expandable member cover 1304. The expandable member cover 1304 can be fabricated from a weave, braid, knit, or membrane, either porous or impermeable to liquids. The expandable member cover 1304 can be affixed to the interior of the expandable member 1302 or to the exterior as illustrated. The expandable member cover 1304 can be deployed inside the expandable member 1302 and be tacked to the expandable member 1302 at a few points or not at all. The points of attachment can be configured to move or slide along the bars of the expandable member 1302 or the points of attachment can be fixed. The expandable member cover 1304 can be elastomeric and biased to self-expand when the expandable member 1302 is expanded. The cover 1304 is illustrated on the distal portion of the expandable member 1302 but the cover can also be positioned on the proximal portion of the expandable member 1302. The amount of expandable member 1302 partial coverage can range from 20% to 75%. The partial expandable member cover 1304 can be beneficial for procedures such as, but not limited to, trapping debris within the expandable member 1302 or for serving as a filter or protection device.

FIG. 13B illustrates a blood vessel 302 comprising an obstruction 308. A microcatheter 1320 has been advanced through the obstruction 308. The microcatheter 1320 comprises the proximal shaft 1310, the expandable region 1302, an exterior mesh cover 1308, and an interior mesh cover 1306.

Referring to FIG. 13B, in a preferred embodiment, the expandable region 1302 would have either an exterior mesh cover 1308 or an interior mesh cover 1306. The exterior mesh cover 1308, or the interior mesh cover 1306, would preferably cover substantially the entire expandable region 1302. In the illustrated embodiment, the exterior mesh cover 1308 is disposed over only the proximal ½ of the expandable region 1302 while the interior mesh cover 1306 is disposed under only the distal ½ of the expandable region. Such a configuration is made here for clarity. Materials suitable for fabricating the interior mesh cover 1306 or the exterior mesh cover 1308 include, but are not limited to, polyurethane, thermoplastic elastomer, silicone elastomer, polyester, polyimide, polyamide, PEEK, PEN, PTFE, or the like. The microcatheter 1310 comprising the full expandable region cover 1306 or 1308 is suitable for partial or complete occlusion of a vessel during a procedure for purposes such as, but not limited to, flow reversal, stagnation generation, and the like. In yet another embodiment, the mesh coating or cover 1306 or 1307 can be disposed along the central, substantially uniform diameter part of the expandable region 1302 but not extend substantially onto the tapered end sections of the expandable region 1302. In this embodiment, blood can continue to flow through the center of the expandable region 1302 and through the tapered ends from, and into, the parent vessel 302, while the cover 1306 or 1307 can serve to completely, or partially, block the neck or entrance to an aneurysm 808 such as that illustrated in FIG. 12C. Such a device can be brought to bear quickly to prevent additional hemorrhage from a ruptured aneurysm on an emergency basis, for example. Furthermore, instrumentation can be introduced through the lumen of the microcatheter to perform therapy distal to the expandable region.

The above presents a description of the devices and methods contemplated for carrying out the present neurointervention and methods of providing said neurointervention, and of the manner and process of making and using the devices, in such full, clear, concise, and exact terms as to enable any person skilled in the art to which it pertains to make and use these neurointerventional devices and methods. These devices and methods are, however, susceptible to modifications and alternate constructions from that discussed above that are fully equivalent. Consequently, these devices and methods are not limited to the particular embodiments disclosed. On the contrary, these devices and methods cover all modifications and alternate constructions coming within the spirit and scope of the devices and methods are as generally expressed by the following claims, which particularly point out and distinctly claim the subject matter of these devices and methods. For example, any element or attribute of one embodiment or example may be incorporated into or used with another embodiment or example, unless otherwise specified of if to do so would render the embodiment or example unsuitable for its intended use. Also, where the steps of a method or process have been described or listed in a particular order, the order of such steps may be changed unless otherwise specified or unless doing so would render the method or process unworkable for its intended purpose. All reasonable additions, deletions, modifications and alterations are to be considered equivalents of the described examples and embodiments and are to be included within the scope of the following claims.

What is claimed is:

1. A catheter device comprising:
a proximal shaft member having a proximal end and a distal end;
a distal shaft member having a proximal end and a distal end;
an expandable member having a distal end connected to the proximal end of the distal shaft member and a proximal end connected to the distal end of the proximal shaft member; and
a variable-length member that extends through the expandable member and is transitionable between a) a short configuration having a first axial length and b) a long configuration having a second axial length longer than said first axial length;
the expandable member assuming an expanded configuration when the variable-length member is in its short configuration and a contracted configuration when the variable length member is in its short configuration.

2. A catheter device according to claim 1 wherein the variable-length member has a curved shape when in its short configuration and a straight or substantially straight shape when in its long configuration.

3. A catheter device according to claim 2 wherein the variable-length member has a plurality of curves when in its short configuration.

4. A catheter device according to claim 2 wherein the variable-length member is helical when in its short configuration.

5. A catheter device according to claim 2 wherein the variable-length member is sinusoidal when in its short configuration.

6. A catheter device according to claim 2 wherein the variable-length member is:
biased to the curved shape/short configuration; and
configured to receive a straightening member which overcomes the bias, causing the variable-length member to transition from the curved shape/short configuration to the straight or substantially straight shape/long configuration.

7. A catheter device according to claim 6 wherein the variable-length member has a lumen for receiving the straightening member.

8. A catheter device according to claim 7 wherein the proximal shaft member has a lumen and wherein the lumen of the variable-length member is aligned with or continuous with the lumen of the proximal shaft member such that the straightening member may advance from the lumen of the proximal shaft member into the lumen of the variable-length member.

9. A catheter device according to claim 8 further comprising a straightening member sized to advance through the lumen of the proximal shaft member and into the lumen of the variable-length member.

10. A catheter device according to claim 9 wherein the straightening member comprises a guidewire.

11. A catheter device according to claim 10 wherein the distal shaft member has a lumen that is aligned or continuous with the lumen of the variable-length member such that the guidewire may further extend into or through the distal shaft member.

12. A catheter device according to claim 11 wherein the distal shaft member has an open distal end so that the guidewire may extend out of the distal end of the distal shaft member.

13. A catheter device according to claim 1 wherein the variable-length member comprises a spring that has a contracted configuration when in its short length and an extended configuration when in its extended length.

14. A catheter device according to claim 13 wherein the spring comprises a coil spring.

15. A catheter device according to claim 13 wherein the variable-length member is configured to receive a spring extending member that causes the spring to move from its contracted configuration to its extended configuration.

16. A catheter device according to claim 15 wherein the variable-length member has a lumen for receiving the spring extending member.

17. A catheter device according to claim 16 wherein the proximal shaft member has a lumen and wherein the lumen of the variable-length member is aligned with or continuous with the lumen of the proximal shaft member such that the spring extending member may advance from the lumen of the proximal shaft member into the lumen of the variable-length member.

18. A catheter device according to claim 17 wherein the lumen of the variable-length member has an engagement surface located distal to the spring such that a distal end of the spring extending member will engage the engagement surface and, thereafter, further advancement of the spring extending member will cause the spring to extend.

19. A catheter device according to claim 17 further comprising a spring engaging member sized to advance through the lumen of the proximal shaft member and into the lumen of the variable-length member.

20. A catheter device according to claim 19 wherein the spring extending member comprises a guidewire.

21. A catheter device according to claim 1 further comprising a locking member for locking the expandable member in at least one of said expanded and contracted configurations.

22. A catheter according to claim 6 further comprising a locking hub on the proximal end of the proximal shaft member useable to lock the straightening member in a desired position.

23. A catheter device according to claim 15 further comprising a locking hub on the proximal end of the proximal shaft member useable to lock the spring extending member in a desired position.

24. A catheter device according to claim 1, wherein the expandable member is sufficiently porous to allow blood to flow past the expandable member when the expandable member is in an expanded configuration.

25. A catheter device according to claim 1 having at least one lumen through which a diagnostic or therapeutic substance may be delivered.

26. A catheter device according to claim 1 having at least one lumen through which a diagnostic or therapeutic device may be advanced.

27. A catheter device according to 25 wherein the lumen has an opening within the expandable member so that substance delivered through the lumen may flow out of the opening within the expandable member.

28. A catheter device according to 25 wherein the lumen has an opening within the expandable member so that a device advanced through the lumen may advance out of the opening within the expandable member.

29. A catheter device according to claim 27 wherein the expandable member is configured such that, when in its expanded configuration, there will exist at least one opening in the expandable member through which substance may flow through the expandable member.

30. A catheter device according to claim 27 wherein the expandable member is configured such that, when in its expanded configuration, there will exist at least one opening in the expandable member through which a device may advance through the expandable member.

31. A system comprising a catheter device according to claim 30 further in combination with an elongate working device that is advanceable through the lumen, out of the lumen opening and through the opening in the expandable member.

32. A system according to claim 31 wherein the elongate working device comprises a device for delivering an embolic device or substance.

33. A system according to claim 32 wherein the elongate working device comprises an embolic coil delivery catheter.

34. A system according to claim 32 wherein the elongate working device is curved to facilitate its advancement out of the lumen opening and through the opening in the expandable member.

35. A catheter device according to claim 1 wherein the expandable member comprises a mesh.

36. A catheter device according to claim 1 having two or more regions of different flexibility.

37. A catheter device according to claim 32 wherein the distal shaft portion is less flexible than the proximal shaft portion.

38. A catheter device according to claim 1 wherein the variable-length member is caused to transition between its short configuration and its long configuration by insertion or movement of an apparatus selected from the group consisting of: a guidewire, a linkage, a pushrod, a push-pull rod.

39. A catheter device according to claim 1 wherein the catheter further comprises an outer shaft and wherein the distal shaft portion slidably fits inside the outer shaft and moves with the distal end of the expandable region.

40. A catheter according to claim 1, wherein the wherein the catheter further comprises an outer shaft and wherein the distal shaft member is connected to the outer shaft by a coupler capable which changes in length in response to longitudinal application of force.

41. A catheter device according to claim 1 sized and configured to advance transluminally into the cerebrovasculature to at least the region of the carotid siphon.

42. A catheter device according to claim 1 sized and configured to advance transluminally into the cerebrovasculature to at least the region of M1 in the middle cerebral artery.

43. A catheter device according to claim 1, wherein the expandable member comprises plurality of longitudinally disposed bars, which are separated from adjacent bars by longitudinally oriented spaces when the expandable member is in its expanded configuration.

44. A method of performing therapy within the cerebrovasculature of a patient comprising:
advancing a guidewire and guide catheter system into the cerebrovasculature from a percutaneous access point in the femoral or iliac arteries;
removing the guidewire from the guide catheter system;
inserting a guidewire into the lumen of a microcatheter to collapse a mesh near the distal end of the microcatheter;
advancing a microcatheter through the guide catheter to a target region within the cerebrovasculature;
withdrawing the guidewire from the microcatheter to expand the mesh within a cerebrovasculature; and
performing therapy or diagnosis within the cerebrovasculature.

45. A method according to claim 44, wherein the therapy or diagnosis comprises advancing a therapeutic or diagnostic catheter through a lumen of the microcatheter.

46. A method according to claim 45, further comprising:
removing the therapeutic or diagnostic catheter from the microcatheter;
inserting the guidewire back into the microcatheter to diametrically collapse the mesh; and
removing the microcatheter from the cerebrovasculature.

47. A method according to claim 45 further comprising removing the therapeutic or diagnostic catheter from the microcatheter.

48. A method according to claim 45 further comprising deploying embolic material from the therapeutic catheter.

49. A method according to claim 45 further comprising deploying embolic coils from the therapeutic catheter.

50. A method according to claim 45 further comprising deploying an embolic mass from the therapeutic catheter, wherein the embolic mass comprises a solvent that is absorbed by the body resulting in hardening of dissolved materials therein.

51. A method according to claim 44 further comprising entrapping thrombus material within the mesh.

52. A method according to claim 51 further comprising diametrically collapsing the mass of thrombus materials entrapped within the mesh.

53. A method according to claim 52 further comprising withdrawing the collapsed thrombus material at least partially into the guide catheter.

54. A method according to claim 53 further comprising expanding a distal portion of the guide catheter to facilitate entrapment of the thrombus material.

55. A method according to claim 54 further comprising removing the guide catheter and the microcatheter from the vasculature of the patient.

56. A catheter according to claim 1 wherein the proximal shaft member and the distal shaft member are integral to each other.

57. A catheter according to claim 1 wherein the proximal shaft member and the distal shaft member are comprised by the same axially elongate structure.

58. A catheter according to claim 1 wherein the proximal shaft member and the distal shaft member are affixed to each other, further wherein the central lumen of the proximal shaft member and the central lumen of the distal shaft member are operably connected.

59. A catheter according to claim 1 wherein the distal shaft member and the variable length member are the same axially elongate structure.

60. A catheter according to claim 1 wherein the proximal shaft member and the variable length member are the same axially elongate structure.

61. A catheter according to claim 1 wherein the distal shaft member and the variable length member are integral to each other and further wherein the central lumens of the distal shaft member and the variable length member are operably connected.

62. A catheter according to claim 1 wherein the proximal shaft member and the variable length member integral to each other and further wherein the central lumens of the proximal shaft member and the variable length member are operably connected.
